(19) United States
(12) Patent Application Publication
(10) Pub. No.: US 2026/0021575 A1
(43) Pub. Date: Jan. 22, 2026

(54) ARTICULABLE MECHANISMS AND RELATED DEVICES AND METHODS

(71) Applicant: INTUITIVE SURGICAL OPERATIONS, INC., Sunnyvale, CA (US)

(72) Inventors: Erik Nelson, Durango, CO (US); Matthew Aaron Wixey, Trumbull, CT (US)

(73) Assignee: INTUITIVE SURGICAL OPERATIONS, INC., Sunnyvale, CA (US)

(21) Appl. No.: 19/266,405

(22) Filed: Jul. 11, 2025

Related U.S. Application Data
(60) Provisional application No. 63/673,851, filed on Jul. 22, 2024.

(51) Int. Cl. B25J 9/10; A61B 34/00; A61B 34/30; B25J 9/00

(52) U.S. Cl. CPC B25J 9/106; A61B 34/30; A61B 34/71; B25J 9/0012; A61B 2034/305

(57) ABSTRACT

An articulable mechanism includes a first link, a second link, and a joint structure coupling the first and second links to each other. The joint structure is configured to enable articulation of the first and second link relative to each other. A first pair of guide channels is defined by and extends through the first link. A second pair of guide channels is defined by and extends through the second link. A lining overlays one or more of at least a portion of the joint structure, the first guide channels, or the second guide channels. A pair of actuation elements extends respectively through the first guide channels, past the joint structure, and through the second guide channels. The lining provides an interior guide surface along at least part of a path of the actuation elements extending through the first and second guide channels and past the joint structure.

### CROSS REFERENCE TO RELATED APPLICATIONS

This application claims the benefit of U.S. Provisional Patent Application No. 63/673,851, filed on Jul. 22, 2024, which is incorporated by reference herein in its entirety.

### TECHNICAL FIELD

Aspects of the present disclosure relate to instruments having articulable mechanisms incorporating joint structures, such as wrists, and related devices, systems, and methods. In particular, aspects of the present disclosure relate to instruments with joint structures that are articulable in response to forces transmitted by actuation elements extending through links coupled together at joints.

### INTRODUCTION

Various instruments, such as medical (e.g., surgical) or other industrial instruments, include articulable mechanisms including joint structures that impart one or more degrees of freedom of movement to such instruments. These articulable mechanisms can include one or more joint structures, each of which can articulate in one or more degrees of freedom, which can be the same or different.

Articulation can be controlled by one or more actuation elements, such as, for example, cables, coupled through various components to a manipulator system that receives inputs from a user, such as a surgeon or other operator, to orient portions of the instrument as desired. Such manipulator systems can include a teleoperated (e.g., computer-controlled) manipulator system. In other applications, the instrument and articulable mechanism can be actuated manually. In some cases, it is desirable that an articulable mechanism exhibits a relatively high stiffness in any given articulation position to facilitate the instrument's ability to maintain a given position under reaction forces, such as those resulting from operation of a working end component, or end effector, of the instrument.

An instrument can include one or more articulable mechanisms, which can be used to couple an end effector coupled to the distal end portion of a shaft of the instrument so as to provide orientation in one or two-degrees of freedom (e.g., pitch and yaw) of the end effector. Other articulable mechanisms can be provided along a length of the shaft to achieve various poses of the shaft of instrument. The one or more actuation elements extend from a transmission mechanism coupled to the shaft proximally of the articulable mechanism, through the instrument shaft and to link(s) of the joint structure(s) of the articulable mechanism. Forces transmitted by the one or more actuation elements impart articulating movement of the link(s) relative to each other about the joint structures, thereby allowing remote “steering” of the articulable mechanism. For example, actuation elements can transmit forces through selective tensioning of flexible, cable-like actuation elements and/or through push/pull forces of stiffer, compressive rod-like actuation elements. Those having ordinary skill in the art have familiarity with various types of actuation elements used to transmit forces to articulate articulable mechanisms.

Because the actuation elements are generally routed in an off-centerline position through a joint structure (i.e., across a joint connecting two links of the articulable mechanism), articulating (bending) an articulable mechanism also results in bending of the actuation element(s). Such repeated bending at the joint structure can lead to changes that affect the integrity of the actuation elements (i.e., reduce cable life), the path length of the actuation element(s), and can impact other characteristics of the articulable mechanism (e.g., wrist), such as the ability of the articulated joint structure to withstand externally applied loads without deflecting from an intended position.

Furthermore, complexities with the fabrication of the components of the joint structure, including fabrication of the joint features making up the joint structure, also contribute to cable degradation and breakage by creating cable guide surfaces (i.e., through the articulable mechanism) with higher than desired friction and having shapes outside of desired design tolerances to achieve optimal joint motion.

There exists a need to provide joint structures for articulable mechanisms in instruments that maximize or improve cable life, without sacrificing the motion quality of the joint structure or increasing the complexity of the joint features. There also exists a need to provide articulatable mechanisms, which address friction and design tolerance issues when routing actuation elements, such as cables, through the joint structure of the mechanism.

### SUMMARY

Exemplary embodiments of the present disclosure may solve one or more of the above-mentioned problems and/or may demonstrate one or more of the above-mentioned desirable features. Other features and/or advantages may become apparent from the description that follows.

In accordance with at least one exemplary embodiment, an articulable mechanism comprises a first link, a second link, and a joint structure coupling the first link and second link to each other. The joint structure is configured to enable articulation of the first link and the second link relative to each other. The articulable mechanism also comprises a first pair of guide channels defined by and extending through the first link and a second pair of guide channels defined by and extending through the second link. The articulable mechanism additionally comprises a lining overlaying one or more of at least a portion of the joint structure, the first pair of guide channels, or the second pair of guide channels. The articulable mechanism further comprises a pair of actuation elements extending respectively through the first pair of guide channels, past the joint structure, and through the second pair of guide channels. The pair of actuation elements extend through the first pair of guide channels and the second pair of guide channels and are configured to be placed in tension to thereby exert a force to control articulation of the first link and the second link relative to each other from a neutral, unarticulated state about the joint structure. The lining provides an interior guide surface along at least part of a path of the pair of actuation elements extending through the first pair of guide channels, the second pair of guide channels, and past the joint structure.

In accordance with at least another exemplary embodiment, a method of manufacturing an articulable mechanism comprises coupling a first link of the articulable mechanism and a second link of the articulable mechanism to each other via a joint structure. The joint structure is configured to enable articulation of the first link and the second link relative to each other. The method also comprises overlaying a lining over one or more of at least a portion of the joint structure, a first pair of guide channels defined by and extending through the first link, or a second pair of guide channels defined by and extending through the second link. The method further comprises extending a pair of actuation elements respectively through the first pair of guide channels, past the joint structure, and through the second pair of guide channels. The lining provides an interior guide surface along at least part of a path of the pair of actuation elements extending through the first pair of guide channels, the second pair of guide channels, and past the joint structure.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are exemplary and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure, and they are incorporated in and constitute a part of this specification. The drawings illustrate one or more exemplary embodiments of the present disclosure and, together with the description, explain certain principles and operation. In the drawings,

FIG. 1 is a schematic view of an embodiment of an instrument comprising an articulable mechanism;

FIG. 2 is a diagrammatic view of an embodiment of a computer-assisted medical system employing robotic technology and including the instrument of FIG. 1;

FIG. 3 is a partial perspective view of an embodiment of a distal end portion of an instrument comprising an articulable mechanism and end effector;

FIG. 4 is a top view of the distal end portion of the instrument of FIG. 3;

FIG. 5 is a partially exploded perspective view of the instrument of FIG. 3 showing actuation elements routed through a joint structure of the articulable mechanism;

FIG. 6 is a perspective view of the instrument of FIG. 3 with a link piece removed to show the actuation elements routed through a distal link of the articulable mechanism and coupled to the end effector;

FIG. 7 is a partial perspective view of another embodiment of a distal end portion of an instrument comprising an articulable mechanism and end effector;

FIG. 8 is a partially exploded perspective view of the instrument of FIG. 7 showing the link pieces and joint structure of the articulable mechanism;

FIG. 9 is an isolated, partial, perspective view of the articulatable mechanism of FIG. 8 illustrating an exemplary lining overlaying each of first and second links coupled by the joint structure, in a neutral, unarticulated state about the joint structure;

FIG. 10 is a partial perspective view of another embodiment of a distal end portion of an instrument comprising an articulable mechanism having a pinned joint structure and an end effector;

FIG. 11 is an isolated, partial, perspective view of an exemplary articulable mechanism having a pinned joint structure, illustrating an exemplary lining overlaying a portion of the joint structure, in an articulated state about the joint structure;

FIG. 12 is an isolated, partial, perspective view of another exemplary articulable mechanism having a pinned joint structure, illustrating another exemplary lining overlaying a portion of the joint structure, in an articulated state about the joint structure;

FIG. 13 is an isolated, partial, perspective view of yet another exemplary articulable mechanism having a pinned joint structure, illustrating an exemplary lining overlaying both a portion of the joint structure and a first link of the joint structure, in an articulated state about the joint structure; and

FIG. 14 is an isolated, partial, perspective view of yet another exemplary articulatable mechanism having a pinned joint structure, illustrating an exemplary lining overlaying a portion of the joint structure and a first link of the joint structure and a second link of the joint structure, in an articulated state about the joint structure.

### DETAILED DESCRIPTION

The present disclosure contemplates articulable mechanisms including joint structures, such as, but not limited to wrists, that include features that can provide desired stiffness and predictable movement, e.g., during articulation, in combination with features to extend the life of actuation elements routed through the joint structure (i.e., which helps maximize or improve cable life during articulation). Contemplated articulable mechanisms may, for example, include features that are configured to reduce friction between the actuation elements and surfaces of the links making up the articulable mechanisms, as the actuation elements pass through the joint structure and move relative to the joint structure during articulation.

In accordance with various embodiments, an articulable mechanism can include a lining overlaying at least a portion of the articulable mechanism, such as, for example, at least one inert that is fitted on a portion of the articulable mechanism, that is configured to modify at least a portion of a path along which a pair of actuation elements extend respectively through a first pair of guide channels defined by and extending through a first link of the articulable mechanism, past the joint structure of the articulable mechanism, and through a second pair of guide channels defined by and extending through a second link of the articulable mechanism. For example, various embodiments of the present disclosure contemplate that the lining overlays one or more of a least a portion of the joint structure, the first pair of guide channels, or the second pair of guide channels. In this manner, the lining may provide an interior guide surface along at least a part of a path of the pair of actuation elements extending through the first pair of guide channels, the second pair of guide channels, and past the joint structure.

In accordance with some embodiments, the lining material is made of a different material than the joint structure, and the first and second links. For example, in some embodiments, the lining is made of a polymeric material, while the components of the articulable mechanism (the links and the joint structure) are made of a metal material. In this manner, the lining provides plastic surfaces over the metal surfaces of the articulable mechanism, over at least a portion of the path of the pair of actuation elements extending through the articulable mechanism (e.g., the lining overlays the first pair of guide channels, a portion of the joint structure itself, and/or the second pair of guide channels) to provide soft and malleable guide surfaces (i.e., in comparison to the metal surfaces of the articulable mechanism itself) along the path of the actuation elements.

In various embodiments, as described further below, the lining can comprise at least one polymeric insert that is fitted over at least a portion of the joint structure, the first pair of guide channels, or the second pair of guide channels. For example, the contemplated inserts can include separate polymeric parts that are configured to be inserted within and/or mechanically interlocked with one or more of the joint structures, the first pair of guide channels, or the second pair of guide channels. In various additional embodiments, as also described further below, the lining can comprise a molded polymeric portion that is applied directly to the articulable mechanism, for example, injection molded directly onto one or more of the parts of the articulable mechanism.

Such polymeric linings (e.g., inserts or molded parts) can, for example, be easily fabricated to include optimal guide channel shapes and geometries (e.g., in either one or both of the first and second links), which may be otherwise difficult to provide during initial fabrication of the metal components forming the joint structure and links of the articulable mechanism. As will be described in more detail below, in accordance with various contemplated methods, such polymeric linings can be either directly molded and/or printed (e.g., via additive manufacturing) onto the components of the articulable mechanism, and/or separately molded and/or printed as discrete parts (e.g., inserts) that are fitted (e.g., friction fit and/or snap fit) onto the components, to provide optimal actuation element guide surfaces based on the design tolerances for a given application. In this manner, the linings may replace various metal surfaces, including various metal actuation element guide surfaces, with plastic surfaces, thereby also functioning to reduce friction between the surfaces and the actuation elements (e.g., which are generally also made of a metal) rubbing on the surfaces of the articulable mechanism.

As discussed above and below, in various exemplary embodiments, articulable mechanisms in accordance with the present disclosure can include a lining that is made of a polymeric material, including a polymer, such as, for example, a medical grade polymer that has been tested and cleared for biocompatibility and safety for medical applications, including, but not limited to medical grade polyethylene, polypropylene, polycarbonate, polyvinyl chloride (PVC), acrylonitrile butadiene styrene (ABS), polystyrene (PS), polyethylene terephthalate glycol (PETG), acrylic (PMMA), and polyether ether ketone (PEEK). Although such polymeric linings have many desirable properties, those of ordinary skill in the art would understand that the contemplated linings can be made from various materials possessing similar properties, such as durability, biocompatibility, non-permeability, sterilization resistance, and lightweightness, and are not limited to being made from a polymeric material.

Thus, by utilizing linings, such as, for example, polymeric linings, that are configured to overlay established joint structures (i.e., having proven motion quality), articulable mechanisms in accordance with the present disclosure can help to increase the life of the actuation elements running through the articulable mechanisms (e.g., providing optimal guide surfaces with reduced friction through the joint structures), without sacrificing the motion quality of the joint structure or increasing the complexity of the joint features of the joint structures.

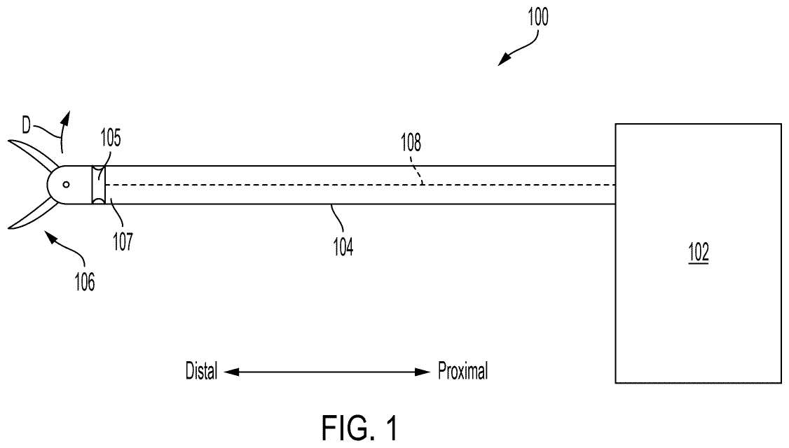

Referring now to FIG. 1, a schematic side view of an embodiment of an instrument 100 (such as, for example, a medical instrument) is shown. The directions “proximal” and “distal” are used herein to define the directions as shown in FIG. 1, with distal generally being in a direction further along a kinematic arm or closest to the worksite in the intended operational use of the instrument 100. While aspects of the present disclosure are discussed in the context of medical or surgical instruments with joint structures in the form of wrists supporting an end effector of the instrument, embodiments of the present disclosure can be used with various instruments used in medical procedures. For example, such instruments include those used for diagnosis, therapy, and sensing, including, for example, imaging instruments such as endoscopes and other imaging instruments. Accordingly, medical instruments as used herein encompasses a variety of instruments used in surgical, diagnostic, and therapeutic applications. In addition, aspects of the disclosure can have non-surgical applications, such as in other remotely-actuatable instruments for inspection and other industrial uses, general robotic uses, manipulation of non-tissue work pieces, etc.

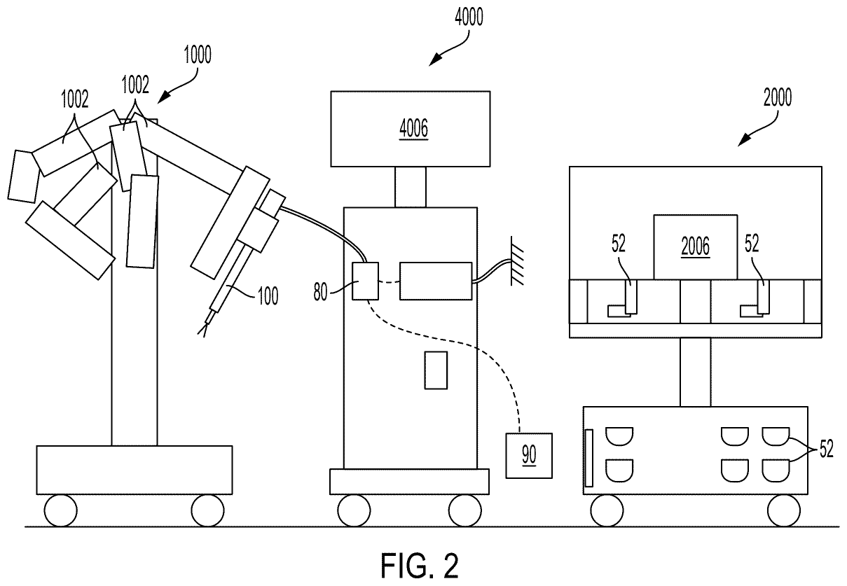

The instrument 100 includes a shaft 104 with a transmission mechanism 102 at a proximal end portion of the shaft 104 and an end effector 106 at a distal end portion 107 of the shaft. In an exemplary embodiment, the transmission mechanism 102 is configured to interface with a manipulating system, such as manipulating systems shown below in connection with FIG. 2. Alternatively, the transmission mechanism 102 can be configured to be operated manually such as for a manual, laparoscopic instrument, which can have a handle or other arrangement configured to be manipulated directly by a user.

The end effector 106 is coupled at the distal end portion 107 of the shaft 104 by an articulable mechanism 105, which can include one or more articulatable joint structures to impart one or more degrees of freedom of movement to the end effector 106 relative to the shaft 104 (for example, to move the end effector 106 in one or more of pitch and yaw). Thus, an articulable mechanism 105 can include two links coupled together by a joint structure, or a series of more than two links coupled by a series of joint structures. For ease and simplification, the embodiments discussed below show two links and a single joint structure referred to below as an articulatable mechanism, but the principles disclosed herein can be applied to articulatable mechanisms that have more than two links and more than one joint structure, as those having ordinary skill in the art would be familiar with. Moreover, articulatable mechanisms in accordance with exemplary embodiments can include a series of links connected with joint structures wherein one or more of the joint structures have the same or different axes about which they articulate the joined links.

Certain coordinated movements of multiple joint structures can enable, for example, articulating the end effector 106 relative to the shaft, longitudinal translations, combined movement in pitch and yaw directions, or other compound movements of the end effector 106 in multiple degrees of freedom relative to the instrument shaft 104. While a single actuation element 108 is shown in connection with FIG. 1, as discussed further below, other, additional actuation elements can also be operably coupled between the transmission mechanism 102 and the articulable mechanism 105 to actuate articulation D of the articulable mechanism 105 along various degrees of freedom associated with individual joint structures.

Operation of the end effector 106 can be controlled by manipulation of the transmission mechanism 102, either manually or through drives of a manipulating system (e.g., the manipulating system shown in FIG. 2). The transmission mechanism 102 includes various mechanical and/or electromechanical devices that transmit motion, energy, and/or signals, e.g., from the manipulating system, or from inputs at the transmission mechanism 102 operable by a user, to the end effector 106. For example, one or more actuation elements (one actuation element 108 shown in FIG. 1) can extend from the transmission mechanism 102, through the shaft 104, and to the end effector 106, to operably couple the transmission mechanism 102 (or a component therein) to the end effector 106. Force applied to the actuation element 108 by the transmission mechanism 102 can actuate (e.g., close, open, or otherwise control) the end effector 106.

While the end effector 106 shown in FIG. 1, and in additional embodiments discussed below, comprises a jaw mechanism including a pair of opposing jaw members, other end effector configurations, such as staplers, clip appliers, ligation tools, and other tools are considered within the scope of this disclosure. Furthermore, an exemplary instrument can similarly include an end effector in the form of an imaging device, such as, for example, an endoscopic camera and the instrument 100 can similarly operate to position a distal end of the camera for viewing of a work site and the operation of surgical instruments within a patient.

In various embodiments, actuation elements can comprise flexible members, such as polymer or metal (e.g., tungsten) solid or braided actuation elements, such as cables. Selective tensioning of the actuation elements can cause transmission of force to the links of an articulatable mechanism to cause articulation in a given direction (i.e., to control articulation of the links relative to each other from a neutral, unarticulated state about the joint structure). Those of ordinary skill in the art are generally familiar with such components and they are thus not described in detail here.

In various embodiments, the transmission mechanism 102 is configured to operably couple to and receive drive inputs from a manipulator system of a teleoperable, computer-assisted medical system that operates at least in part with robotic technology (sometimes referred to as a robotic surgical system). One embodiment of such a computer-assisted medical system is illustrated in the schematic diagram of FIG. 2, depicting a manipulator system 1000 comprising a plurality of manipulator arms 1002 to which the transmission mechanism 102 of instrument 100 can operably couple, a surgeon side console 2000 comprising various master inputs 52 and a surgeon viewer 2006 which can include video images of the remote worksite taken through an endoscopic imaging device, and/or other graphical information, and a vision/control console 4000 which can also have a display 4006 presenting similar images as the viewer 2006 or other information relating to a procedure.

The vision/control console 4000 also can in some embodiments comprise components that supply auxiliary functionality to instruments, such as via an auxiliary unit 80, which can be, for example, insufflation gas, vacuum for evacuation, electrosurgical energy, and similar flux supply units. Such units can be controlled through a controller integrated with the system and/or can be separately controlled at a stand-alone input unit 90, rather than through the surgeon side console 2000. A non-limiting embodiment of a computer-assisted, teleoperable medical system with which the instrument 100 and various instrument embodiments described herein can be utilized are the da Vinci® Surgical Systems commercialized by Intuitive Surgical, Inc., of Sunnyvale, California.

In various other embodiments contemplated within the scope of the present disclosure, the medical instrument 100 can be configured to be manually actuated, with the proximally located, transmission mechanism 102 having inputs that are configured to be manually actuated rather than coupled to a manipulator arm. Yet other embodiments contemplate the instrument can have both manually actuated inputs and inputs configured to be driven by drive outputs of a manipulator system.

As discussed above, the present disclosure contemplates medical instruments (e.g., instrument 100) including articulable mechanisms (e.g., articulable mechanism 105) incorporating joint structures, such as, but not limited to wrist assemblies, which are configured to provide various degrees of rotation relative to an end effector connected to the wrist assembly. Such wrist assemblies can, for example, be similar to those shown and described in International Patent Application Publication No. WO 2023/177565 A1 (filed internationally on Mar. 8, 2023), entitled “Medical Devise Wrist,” and International Patent Application Publication No. WO 2019/173267 A1 (filed internationally on May 3, 2019), entitled “Low-Friction, Small Profile Medical Tools having Easy-to-Assemble Components,” each of which is incorporated herein by reference in its entirety.

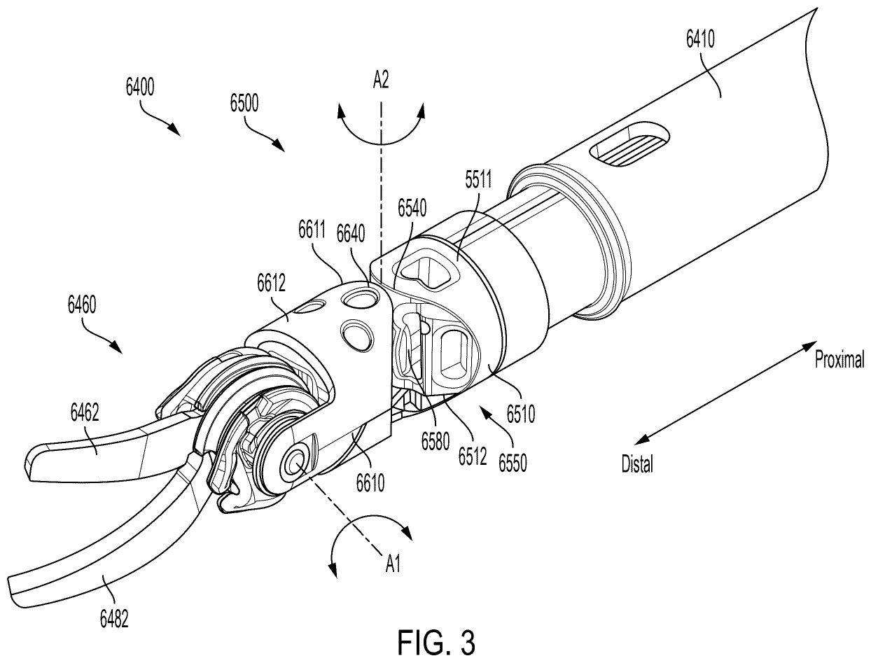

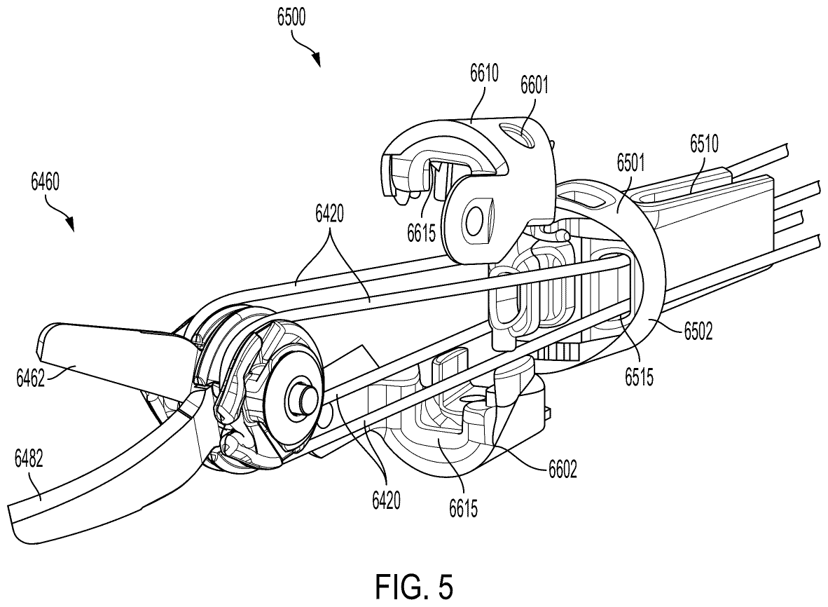

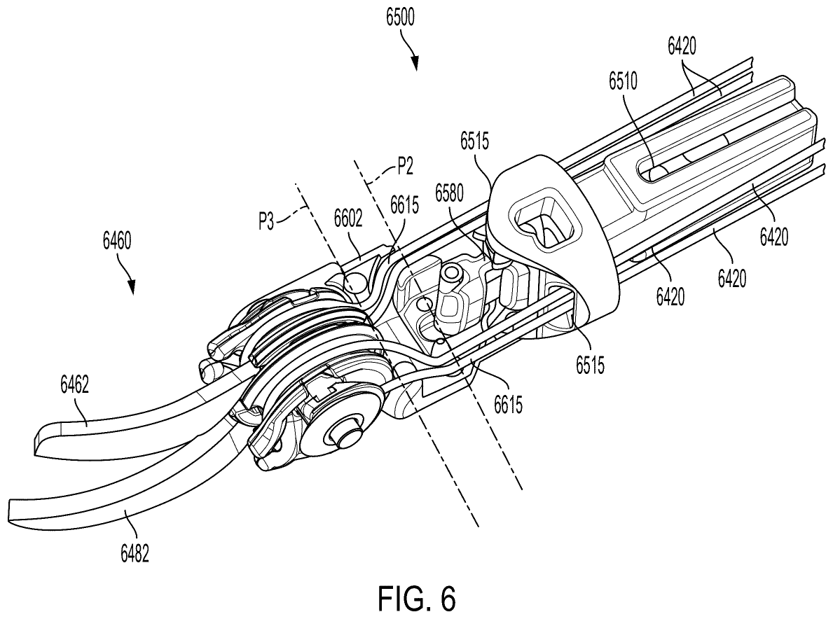

FIGS. 3-6, for example, illustrate an instrument 6400 incorporating an articulable mechanism, in the form of a wrist assembly 6500, in accordance with various embodiments. The instrument 6400 includes a proximal mechanical structure (e.g., transmission mechanism 102), a shaft 6410, a wrist assembly 6500, an end effector 6460, and a set of actuation elements (see FIGS. 5 and 6 identifying the actuation elements as cables 6420). As discussed above, the actuation elements (e.g., cables 6420) function as tension elements that couple the proximal mechanical structure to the wrist assembly 6500 and end effector 6460. The instrument 6400 is configured such that movement of one or more of the cables produces rotation of the end effector 6460 about a first axis of rotation A1 (see FIG. 3, which may function as a yaw axis), rotation of the wrist assembly 6500 about a second axis of rotation A2 (see FIG. 3, which may function as a pitch axis), a cutting rotation of tool members 6462 and 6482 of the end effector 6460 (e.g., which is in the form of a jaw mechanism) about the first axis of rotation A1, or any combination of these movements.

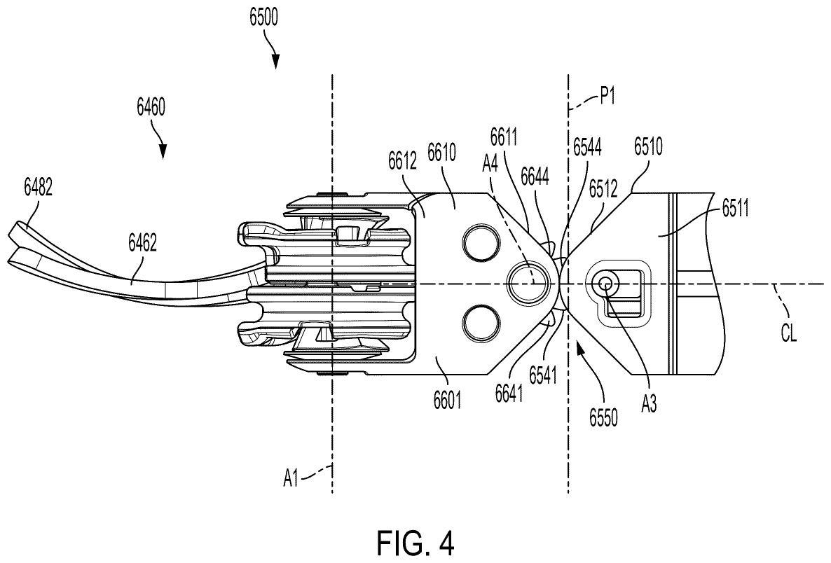

The wrist assembly 6500 includes a first link 6510 (which functions as a proximal wrist link), a second link 6610 (which functions as a distal wrist link), and a joint structure 6550 coupling the first link 6510 and the second link 6610 to each other, such that the joint structure 6550 is configured to enable articulation of the first link 6510 and the second link 6610 relative to each other. In some embodiments, the wrist assembly 6500 also includes a connector link 6580. The connector link 6580 is coupled, for example, between the first link 6510 and the second link 6610 to form the articulating wrist assembly 6500. In some embodiments, the first link 6510 is coupled to the connector link 6580 via a pinned joint such that the first link 6510 is rotatable with reference to the connector link 6580 about a third rotation axis A3, which functions as a proximal connector link rotation axis (see FIG. 4). The second link 6610 is also coupled to the third link 6580 via a pinned joint such that the second link 6610 is rotatable with reference to the connector link 6580 about a fourth rotation axis A4, which functions as a distal connector link rotation axis (see FIG. 4). Because the joint between the first link 6510 and the second link 6610 is not a pinned joint, the second rotation axis A2 can move relative to the first link 6510 (i.e., during rotation of the second link 6610) in a snake-like manner.

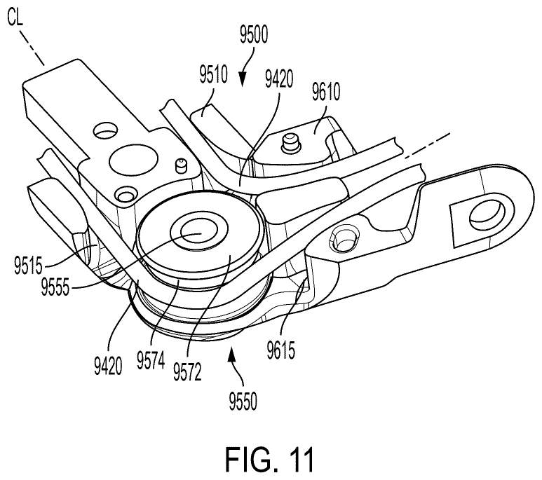

As illustrated in FIG. 4, for example, which shows the articulable mechanism in a neutral, unarticulated state about the joint structure, the wrist assembly 6500 defines a longitudinal centerline CL. Although the longitudinal centerline CL is shown as being linear when the articulable mechanism is in the neutral, unarticulated state, when the wrist assembly is moved into different orientations to place the articulable mechanism in an articulated state (i.e., when the second link 6610 rotates relative to the first link 6510), the longitudinal centerline CL can be curved (see FIG. 11).

The first link 6510 has a proximal portion 6511 and a distal portion 6512, and the second link 6610 has a proximal portion 6611 and a distal portion 6612. The proximal portion 6611 is rotatably coupled to the distal portion 6512 of the first link 6510. As described herein, the second link 6610 rotates relative to the first link 6510 about the second rotation axis A2, and the distal portion 6612 is coupled to the end effector 6460. In various embodiments, the second link 6610 includes a discrete first link piece 6601 and a discrete second link piece 6602 (see the exploded view of FIG. 5). In this manner, in such embodiments, the first link piece 6601 and the second link piece 6602 can be constructed as separate pieces and later coupled together to form the second link 6610. By forming the second link 6610 from two discrete pieces, the method of assembly of the instrument 6400 can be made more efficient than that for a device with a monolithically constructed wrist link. For example, as described further below, the actuation elements (e.g., the cables 6420) can be placed into cable guide channels 6615 before the second link piece 6602 is coupled to the first link piece 6601, thereby eliminating the need to pass loose ends of the cables 6420 through enclosed channels. The second link piece 6602 can be coupled to the first link piece 6601 by any suitable mechanism (e.g., an adhesive joint, a weld joint, or a mechanical fastener), as would be understood by those of ordinary skill in the art.

In some embodiments, the first link piece 6601 and the second link piece 6602 can have the same configuration, such that the first link piece 6601 and the second link piece 6602 are substantially identical pieces, while in other embodiments, the first link piece 6601 and the second link piece 6602 can have different configurations. Moreover, although not described in detail separately herein, in a similar manner, in various embodiments, the first link 6510 includes a discrete first link piece 6501 and a discrete second link piece 6502

With reference to FIGS. 3 and 4, to enable timed articulation between the first link 6510 and the second link 6610 the joint structure 6550 of the wrist assembly 6500 may further include one or more joint features, including, for example, complementary rolling contact surfaces and/or complementary and intermeshing joint features. In various embodiments, for example, the distal portion 6512 of the first link 6510 includes a joint feature 6540 that is rotatably coupled to a mating joint feature 6640 of the proximal portion 6611 of the second link 6610. For example, the joint feature 6540 includes a first set of teeth 6541, a second set of teeth positioned diametrically opposite to the first set of teeth (not shown in the top view of FIG. 4), and curved contact surfaces 6544. The first set of teeth 6541 intermesh with a corresponding first set of teeth 6641 on the second link 6610 and the second set of teeth intermesh with a corresponding second set of teeth (not shown) on the second link 6610. In this manner, when the second link 6610 rotates relative to the first link 6510 (i.e., pitch rotation about the second rotation axis A2), the curved contact surfaces 6544 are in rolling contact with the corresponding curved contact surfaces 6644 of the second link 6610. As those of ordinary skill in the art would be familiar with the different types of mating joint features that can be incorporated into such joint structures to enable articulation of the first link and the second link relative to each other, such joint features will not be discussed in further detail herein.

Articulable mechanisms in accordance with the present disclosure can, for example, incorporate joint structures having joint features (e.g., mating joint features 6540 and 6640) similar to those shown and described in U.S. Pat. No. 10,639,805 (issued May 5, 2020), entitled “Mechanical Wrist Joints with Enhanced Range of Motion, and Related Devices and Methods,” which is incorporated herein by reference in its entirety. Those of ordinary skill in the art will further understand that the wrist assembly 6500, and mating joint features 6540 and 6640, described herein and illustrated in FIGS. 3-6 are exemplary only.

In some embodiments, the wrist assembly 6500 is formed from a metallic material. Metallic joint structures provide strength to the articulable mechanism, while also allowing the delivery of electrical energy to the end effector 6460 (e.g., the tool members 6462 and 6482). For example, as illustrated in FIGS. 5 and 6, each of the first link 6510 and the second link 6610 can be formed from a metallic material, with the first link 6510 defining a first pair of guide channels 6515 extending through the first link 6510 and the second link 6610 defining a second pair of guide channels 6615 extending through the second link 6610 (e.g., each pair of guide channels 6515 and 6615 extending along the longitudinal centerline CL). Thus, as disclosed in International Patent Application Publication No. WO 2023/177565 A1, incorporated by reference herein, such articulable mechanisms, including, for example, the wrist assembly 6500, can be referred to as pulley-less articulable mechanisms. Additionally, as illustrated best perhaps in the exploded view of FIG. 5, there is no central structure or guide surface within the wrist assembly 6500 that is between the first link 6510 and the second link 6610 as the cables 6420 pass through the wrist assembly 6500.

Accordingly, in such articulable mechanisms, it is desirable to shape and contour the guide channels to produce the desired path for the actuation elements (e.g., cables 6420) to pass therethrough. For example, it may be desirable to shape and contour the side walls of the first link 6510 and the second link 6610 (i.e., which respectively define the guide channels 6515 and the guide channels 6615) to provide the desired friction and bending characteristics for routing the actuation elements (e.g., cables 6420) therethrough. Due to the complexity of the links 6510 and 6610, which may be formed of by discrete link pieces 6501, 6601 and 6502, 6602 that also include intricate joint features as discussed above, it may be difficult, however, to fabricate guide channels (i.e., directly within the links 6510 and 6610) that have optimal friction and bending characteristics, thereby resulting in cable guide surfaces with higher then desired friction and having shapes that are outside of desired design tolerances. To provide optimal interior guide surfaces for the actuation elements (e.g., cables 6420) extending across the joint structure (i.e., between the links 6510 and 6610), various embodiments of the present disclosure further contemplate providing a lining over one or more of at least a portion of the joint structure (e.g., 6550), the first pair of guide channels (e.g., 6515), or the second pair of guide channels (e.g., 6615).

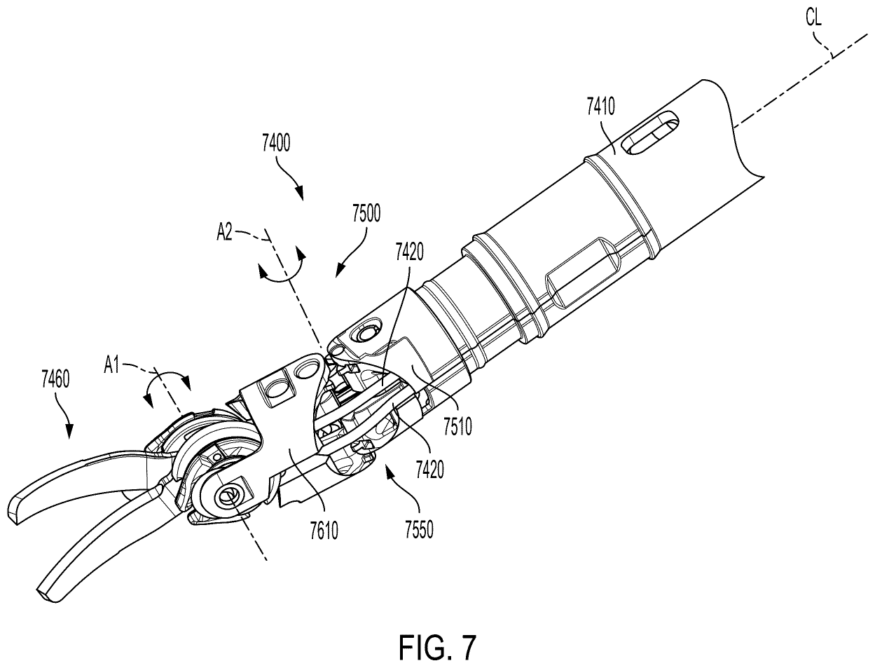

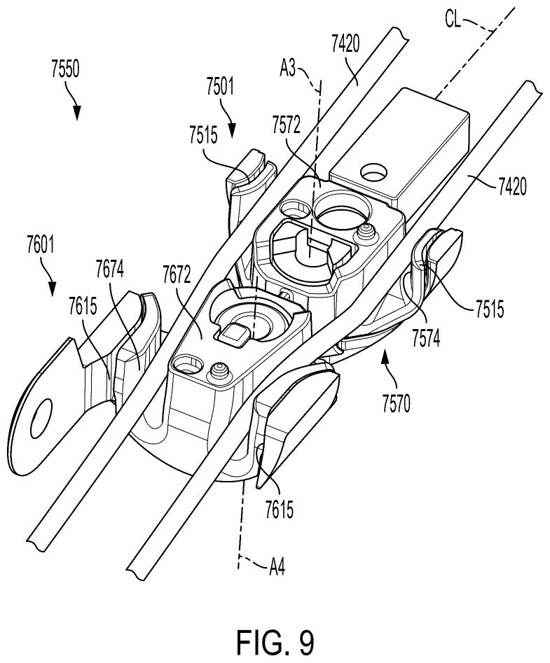

FIGS. 7-9 illustrate, for example, another embodiment of a distal end portion of an instrument 7400 comprising a pulley-less articulable mechanism, in the form of a similar wrist assembly 7500, including a lining in accordance with the present disclosure. Similar to the instrument 6400, the instrument 7400 includes a proximal mechanical structure (e.g., transmission mechanism 102), a shaft 7410, a wrist assembly 7500, an end effector (e.g., in the form of a jaw mechanism 7460), and a set of actuation elements (see FIG. 9 identifying the actuation elements as cables 7420).

The wrist assembly 7500 includes a first link 7510 (which functions as a proximal wrist link), a second link 7610 (which functions as a distal wrist link), and a joint structure 7550 coupling the first link 7510 and the second link 7610 to each other, such that the joint structure 7550 is configured to enable articulation of the first link 7510 and the second link 7610 relative to each other. For example, in various embodiments, to enable timed articulation between the first link 7510 and the second link 7610 the joint structure 6550 may include a first joint feature 7540 fixed to the first link 7510 and a second joint feature 7640 fixed to the second link 7610. For example, the joint features 7540 and 7640 may include complementary rolling contact surfaces and/or complementary and intermeshing joint features, as discussed above. As the joint structure, joint features, and movements of the wrist assembly 7500 are similar to the structure, joint features, and movements of the wrist assembly 6500, discussed above with reference to the embodiment of FIGS. 3-6, these features will not be discussed in further detail.

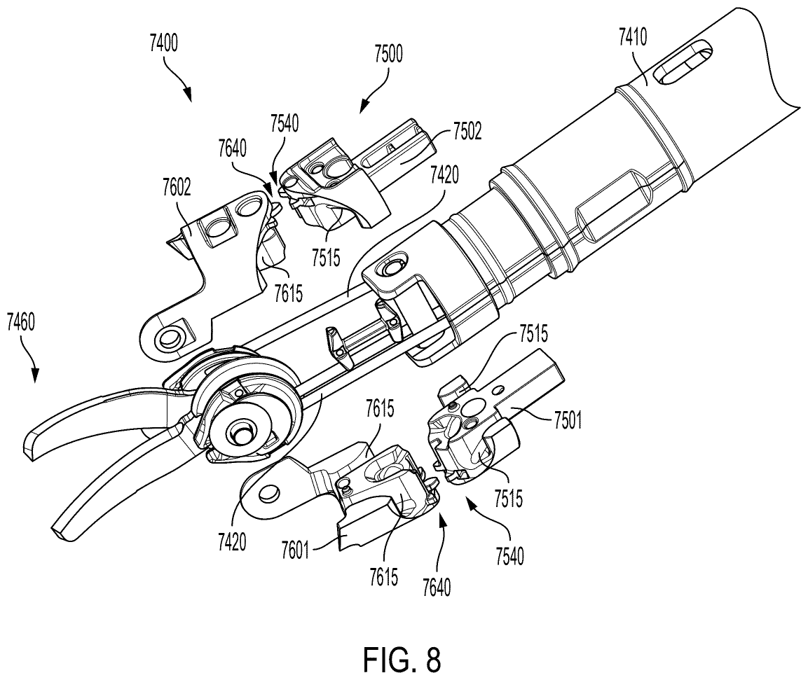

Also similar to the above wrist assembly 6500, as perhaps best illustrated in the exploded view of FIG. 8, in various embodiments, the first link 7510 includes a discrete first link piece 7501 and a discrete second link piece 7502, and the second link 7610 includes a discrete first link piece 7601 and a discrete second link piece 7602. In this manner, in such embodiments, the first link piece 7501 and the second link piece 7502 can be constructed as separate pieces and later coupled together to form the first link 7510, and the first link piece 7601 and the second link piece 7602 can be constructed as separate pieces and later coupled together to form the second link 7610 (as illustrated in FIG. 7).

In such embodiments, each of the first and second link pieces 7501 and 7502 includes a portion of a first pair of guide channels 7515, such that when the link pieces 7501 and 7502 are coupled together (i.e., to form the first link 7510), the first pair of guide channels 7515 is defined by and extends through the first link 7510. Similarly, each of the first and second link pieces 7601 and 7602 includes a portion of a second pair of guide channels 7615, such that when the link pieces 7601 and 7602 are coupled together (i.e., to form the second link 7610), the second pair of guide channels 7615 is defined by and extends through the second link 7610. As shown for example in the isolated view of FIG. 9, which depicts only the first link pieces 7501 and 7601, the channels 7515 of the first pair of guide channels 7515 are on opposite sides of an articulation axis A3 (see, also, FIG. 4) of the first link 7510 about the joint structure 7550 and the channels 7615 of the second pair of guide channels 7615 are on opposite sides of an articulation axis A4 (see, also, FIG. 4) of the second link 7610 about the joint structure 7550. In this manner, a first pair of actuation elements (e.g., cables 7420) can extend respectively through the first pair of guide channels 7515, past the joint structure 7550, and through the second pair of guide channels 7615. Although not depicted in detail for ease of illustration, those of ordinary skill in the art would understand that, in a similar manner, a second pair of actuation elements (e.g., cables 7420) can extend through the second link pieces 7502 and 7602 of the links 7510 and 7610 (see FIGS. 7 and 8), such that the second pair of actuation elements also extend respectively through the first pair of guide channels 7515, past the joint structure 7550, and through the second pair of guide channels 7615.

The wrist assembly 7500 also includes a lining 7570 overlaying portions of the joint structure 7550, the first pair of guide channels 7515, and the second pair of guide channels 7615. As illustrated in the partial view of FIG. 9, in some embodiments, the lining 7570 can include inserts 7572 and 7672, respectively fitted over each of the first link pieces 7501 and 7601. In this manner, the insert 7572 is fitted over at least a portion of the first pair of guide channels 7515 and the insert 7672 is fitted over at least a portion of the second pair of guide channels 7615, such that the inserts 7572 and 7672 together provide an interior guide surface through the articulable mechanism. For example, the insert 7572 provides an interior guide surface 7574 along a first portion of a path of the pair of cables 7420 (i.e., through the first link 7510), and the insert 7672 provides an interior guide surface 7674 along a second portion of the path of the pair of cables 7420 (i.e., through the second link).

Although also not depicted for ease of illustration, those of ordinary skill in the art would further understand that, in a similar manner, in additional embodiments, the lining 7570 can include additional inserts fitted over each of the second link pieces 7502 and 7602, such that when the first link pieces 7501, 7502 are coupled together (i.e., to form the first link 7510) and the second link pieces 7601, 7602 are coupled together (i.e., to form the second link 7610), each of the first pair of guide channels 7515 and the second pair of guide channels 7615 is fully lined by the respective inserts to provide interior guide surfaces for both the first and second pairs of actuation elements (e.g., cables 7420). In this manner, in such embodiments, the inserts lining the respective link pieces (i.e., lining each of the first link pieces 7501, 7502 and each of the second link pieces 7601, 7602) can also be coupled together when the link pieces are coupled together, as would be understood by those of ordinary skill in the art.

As discussed above, in some embodiments, the lining 7570 can include inserts 7572 and 7672 that are molded or printed (e.g., via additive manufacturing) as separate polymeric parts configured to be fit over at least a portion of the joint structure 7550, the first pair of guide channels 7515, or the second pair of guide channels 7615. For example, the inserts 7572 and 7672 can be configured to be mechanically interlocked with at least the portion of the joint structure 7550, the first pair of guide channels 7515, or the second pair of guide channels 7615 via, for example, a snap-fit connection, or can be fitted onto one or more of the components via an interference fit. In some other embodiments, the lining 7570 can instead be molded directly over (i.e., over molded) and bonded to at least a portion of the joint structure 7550, the first pair of guide channels 7515, or the second pair of guide channels 7615, for example, via an injection molding process.

In this manner, the lining 7570 is separately formed from the metal components of the wrist assembly 7500 (e.g. being formed from a different material and using a different process) to specially take into account considerations informing the shape of the first pair of guide channels 7515 and/or the second pair of guide channels 7615 (and any guide channels described herein), including, for example, extending cable life, reducing friction losses (e.g., from cable movement within the channels), maintaining the actuation elements (e.g. cables 7420) within a distal boundary defined for the wrist assembly 7500, minimizing a fleet angle of the cables 7420 with respect to their connection to the end effector 7460, and positioning the cables 7420 relative to a longitudinal centerline CL (see FIG. 7) to maximize the torque that can be applied about one of the axes of rotation of the wrist assembly 7500 or end effector 7460. For example, the guide channels 7515 and 7615 can be shaped to reduce sharp bends, which can help reduce wear on the cables (to extend cable life), while also reducing friction losses when the cables 7420 are moved therein.

Those of ordinary skill in the art will, therefore, understand that that the articulable mechanism in the form of wrist assembly 7500, including the lining 7570 (e.g., comprised of inserts 7572 and 7672), discussed and illustrated with reference to FIGS. 7-9, is exemplary only and that articulable mechanisms in accordance with the present disclosure can have various types of joint structures and links, which utilize various types of linings, formed from various types of materials while utilizing various methods, and which overlay various portions of the articulable mechanism, without departing from the scope of the present disclosure and claims. Indeed, those of ordinary skill in the art would understand that a particular application (e.g., type of articulable mechanism/joint structure), with a defined set of design considerations, may dictate the type and configuration of any given lining.

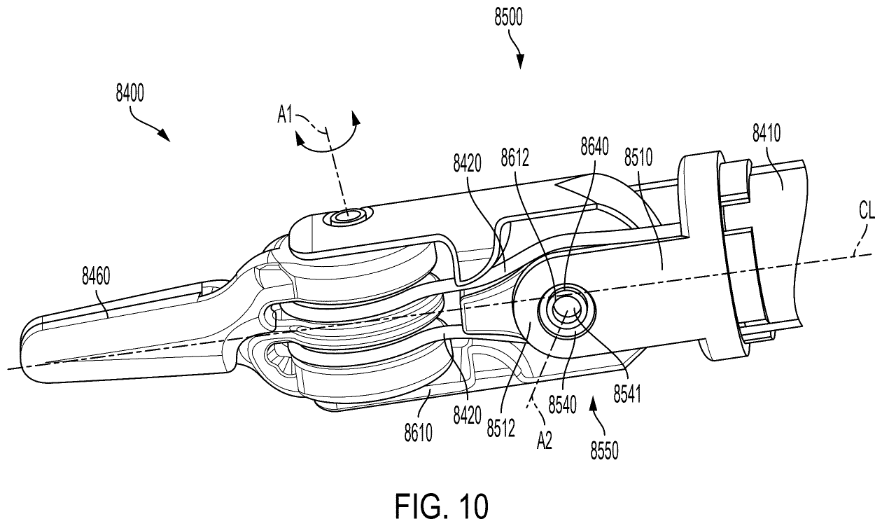

Referring now to FIG. 10, for example, a distal end portion of an instrument 8400 comprising another type of articulable mechanism, in the form of a pinned wrist assembly 8500, which can include various additional exemplary linings, is illustrated. Similar to the instruments 6400 and 7400, the instrument 8400 includes a proximal mechanical structure (e.g., transmission mechanism 102), a shaft 8410, a pinned wrist assembly 8500, a distal end effector (e.g., in the form of a jaw mechanism 8460) that is configured to rotate about a first axis of rotation A1 (which may function as a yaw axis as described above), and a set of actuation elements (see FIG. 10 identifying the actuation elements as cables 8420). The pinned wrist assembly 8500 includes a first link 8510 (which functions as a proximal wrist link), a second link 8610 (which functions as a distal wrist link), and a joint structure 8550 coupling the first link 8510 and the second link 8610 to each other, such that the joint structure 8550 is configured to enable articulation of the first link 8510 and the second link 8610 relative to each other.

In contrast to the above joint structures 6550 and 7550, the joint structure 8550 includes a pinned joint, having a forked end portion 8512 including a joint feature 8540 that is rotatably coupled to a hub structure 8612 of a mating joint feature 8640 of the second link 8610 via, for example, a pin 8541. In this manner, the first link 8510 and the second link 8610 form the pinned wrist assembly 8500, with the hub structure 8612 providing an outer surface along which the actuation elements (e.g., cables 8420) extend across the joint 8550, and having a second axis of rotation A2 (also referred to as the pitch axis) about which the second link 8610 can rotate relative to the first link 8510, with the pin 8541 extending through both the distal end joint portion 8540 and the second link joint portion 8640 to rotatably couple the second link 8610 to the first link 8510. As shown in FIG. 10, the first link 8510 and the second link 8610 define a longitudinal centerline CL that intersects the pitch axis A2 when the instrument 8400 is in a neutral, unarticulated state about the joint structure 8550 (e.g., a “straight” configuration).

Similar to the above wrist assembly 7500, to provide an interior guide surface along at least part of a path of the actuation elements (e.g., cables 8420) across the joint structure 8550, the pinned wrist assembly 8500 can also include a lining overlaying various portions of pinned wrist assembly 8500, including one or more of at least a portion of the joint structure 8550, a first pair of guide channels defined by and extending through the first link 8510, or a second pair of guide channels defined by and extending through the second link 8610. FIGS. 11-14 (which for clarity purposes depict only a first set of link pieces as discussed above in more detail with reference to the embodiments of FIGS. 3-9), illustrate various exemplary configurations of articulable mechanisms incorporating pinned joint structures (e.g., similar to pinned wrist assembly 8500), which include various additional exemplary linings in accordance with the present disclosure.

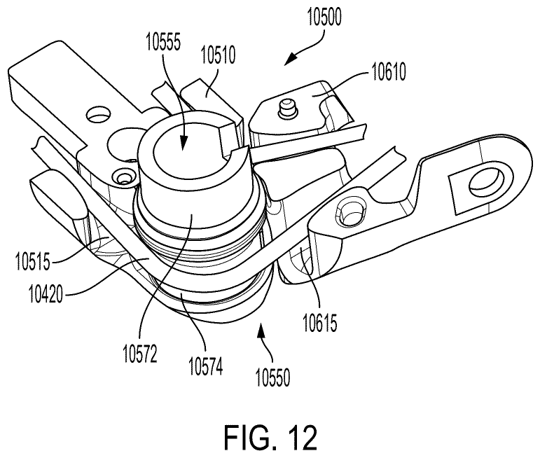

In such embodiments, the contemplated linings may overlay at least a portion of the pinned joint structure, such as, for example, at least a hub structure of the pinned joint structure. With reference to FIGS. 11 and 12, for example, articulable mechanisms 9500, 10500 include pinned joint structures 9550, 10550 coupling a first link 9510, 10510 to a second link 9610, 10610, which have linings overlaying hub structures 9555, 10555 of the pinned joint structures 9550, 10550. As illustrated in the partial views of FIGS. 11 and 12, in some embodiments, the linings may comprise inserts 9572, 10572 that are fitted over the hub structure 9555, 10555, such that the inserts 9572, 10572 provide an outer surface 9574, 10574 along which a pair of actuation elements 9420, 10420 may extend across the joint structures 9550, 10550. In this manner, in such embodiments, the actuation elements 9420, 10420 can extend through a first pair of guide channels 9515, 10515 defined by and extending through the first link 9510, 10510, across the joint structure 9550, 10550 along the outer surface 9574, 10574 of the insert 9572, 10572, and through a second pair of guide channels 9615, 10615 defined by and extending through the second link 9610, 10610.

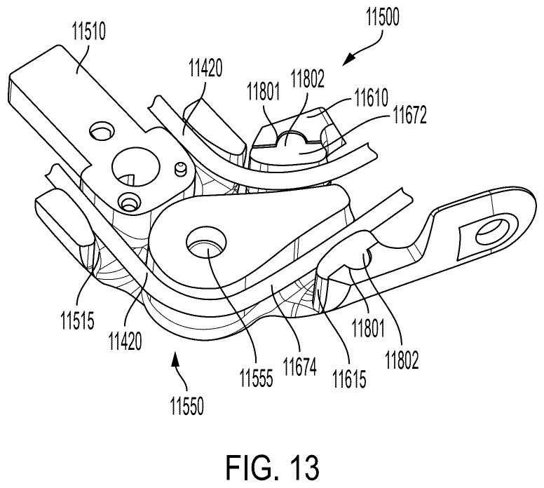

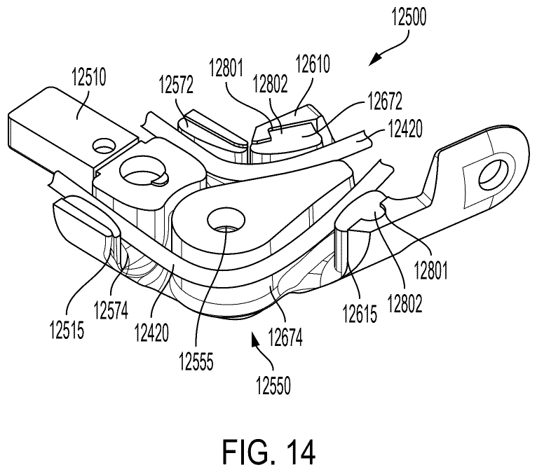

With reference to FIGS. 13 and 14, articulable mechanisms 11500, 12500 include pinned joint structures 11550, 12550 coupling a first link 11510, 12510 to a second link 11610, 12610, which have linings overlaying hub structures 11555, 12555 of the pinned joint structures 9550, 10550 and one or both of a first pair of guide channels 11515, 12515 defined by and extending through the first link 11510, 12510 and a second pair of guide channels 11615, 12615 defined by and extending through the second link 11610, 12610. As illustrated in FIG. 13, in some embodiments, the lining may include a single lining that overlays both the hub structure 11555 and the second pair of guide channels 11615, such as, for example, a single insert 11672 that is fitted over the hub structure 11555 and the guide channels 11615, such that the insert 11672 provides a guide surface 11674 along which a pair of actuation elements 11420 may extend across the joint structure 11550. In this manner, in such embodiments, the actuation elements 11420 can extend through the first pair of guide channels 11515 and along the guide surface 11674 of the insert 11672.

In some additional embodiments, as illustrated in FIG. 14, the lining may include multiple linings, which overlay each of the first pair of guide channels 12515, the hub structure 12555, and the second pair of guide channels 12615. Such as, for example, a first insert 12572 that is fitted over the first pair of guide channels to provide a guide surface through the first link 12510, and a second insert 12672 that is fitted over both the hub structure 12555 and the second pair of guide channels 12615 to provide a guide surface across the joint structure 12550 and through the second link 12610. In this manner, in such embodiments, the actuation elements 12420 can extend along the outer surface 12574 of the first insert 12572 and along the outer surface 12674 of the second insert 12672.

As discussed above, the contemplated linings can include inserts 9572, 10572, 11672, 12572, 12672 that are molded or printed (e.g., via additive manufacturing) as separate polymeric parts configured to be fit over at least a portion of the joint structure, the first pair of guide channels, or the second pair of guide channels. For example, with reference to the embodiments of FIGS. 13 and 14, in some embodiments, the inserts 11672, 12572, 12672 can include male features 11802, 12802 that are configured to matingly engage with corresponding female features 11801, 12801 on the links 11610, 12610 to mechanically interlock the parts together. In some other embodiments, the linings can instead be molded directly over (i.e., over molded) and bonded to at least a portion of the articulable mechanisms 9500, 10500, 11500, 12500, for example, via an injection molding process.

The articulable mechanisms 8500, 9500, 10500, 11500, 12500, and the various linings discussed and illustrated with reference to the embodiments of FIGS. 10-14, are exemplary only and, as discussed above, articulable mechanisms in accordance with the present disclosure can have various types of joint structures and links, which utilize various types and combinations of linings, formed from various types of materials (e.g., polymeric materials) while utilizing various methods (e.g., mechanical interlocking mechanisms, interference fit methods, and/or injection molding methods) to overlay the linings on the mechanisms, and the contemplated linings can overlay various different portions of the articulable mechanisms, without departing from the scope of the present disclosure and claims. Based on a particular application, further modifications and alternative embodiments will be apparent to those of ordinary skill in the art in view of the disclosure herein. Different configurations and arrangements of linings may, for example, be substituted for those illustrated and described herein, parts and processes may be reversed, and certain features of the present teachings may be utilized independently, all as would be apparent to one skilled in the art after having the benefit of the description herein.

The embodiments described herein can be well suited for use in medical applications. In particular, some embodiments are suitable for use in, for example, surgical, teleoperated surgical, diagnostic, therapeutic, and/or biopsy procedures. Such procedures could be performed, for example, on human patients, animal patients, human cadavers, animal cadavers, and portions or human or animal anatomy. Some embodiments can also be suitable for use in, for example, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments can also be used for benchtop procedures on non-living material and forms that are not part of a human or animal anatomy. Moreover, some embodiments are also suitable for use in non-medical applications, such as industrial robotic uses. In non-limiting embodiments, the techniques, methods, and devices described herein can be used in, or can be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein can be embodied and implemented in various ways and systems, including manually operated instruments and computer-assisted, teleoperated systems, in both medical and non-medical applications. Reference to the daVinci® Surgical Systems is illustrative and not to be considered as limiting the scope of the disclosure herein.

As used herein and in the claims, terms such as computer-assisted or teleoperable in referencing manipulator systems, or the like should be understood to refer broadly to any system comprising one or more controllable kinematic structures (“manipulators”) that are movable and controllable at least in part through the aid of an electronic controller (with or without human inputs). Such systems can occasionally be referred to in the art and in common usage as robotically assisted systems or robotic systems. Such systems include systems that are controlled by a user (for example through teleoperation), by a computer automatically (so-called autonomous control), or by some combination of these. In examples in which a user controls at least some of the operations of the manipulator, an electronic controller (e.g., a computer) can facilitate or assist in the operation. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems can be implemented in a variety of contexts to perform a variety of procedures, both medical and non-medical. Thus, although some examples described in greater detail herein can be focused on a medical context, the devices and principles described herein are also applicable to other contexts, such as industrial manipulator systems.

This description and the accompanying drawings that illustrate various aspects and embodiments should not be taken as limiting—the claims define the scope of protection. Various mechanical, compositional, structural, electrical, and operational changes can be made without departing from the spirit and scope of this description and the claims. In some instances, well-known structures, components, and techniques have not been shown or described in detail in order not to obscure the present disclosure. Like numbers in two or more figures that end in the same two digits but begin with a different series, such as 8xx, 10xx, etc. are as best as possible used to represent the same or similar elements.

Elements and their associated aspects that are described in detail with reference to one embodiment can, whenever practical, be included in other embodiments in which they are not specifically shown or described. For example, if an element is described in detail with reference to one embodiment and is not described with reference to a second embodiment, the element can nevertheless be claimed as included in the second embodiment.

Further, this description's terminology is not intended to be limiting. For example, spatially relative terms—such as “beneath”, “below”, “lower”, “above”, “upper”, “proximal”, “distal”, and the like—can be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device can be otherwise oriented (rotated 90 degrees or at other orientations) and the spatially relative descriptors used herein interpreted accordingly. Likewise, descriptions of movement along and around various axes include various special device positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled can be electrically or mechanically directly coupled, or they can be indirectly coupled via one or more intermediate components. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Other embodiments in accordance with the present disclosure will be apparent to those skilled in the art from consideration of the specification and figures, and practice of the embodiments disclosed herein. It is intended that the specification and embodiments be considered as illustrative only, with the following claims being entitled to their fullest breadth, including equivalents, under the applicable law.

What is claimed is:

1. An articulable mechanism, comprising:
a first link;
a second link;
a joint structure coupling the first link and the second link to each other, wherein the joint structure is configured to enable articulation of the first link and the second link relative to each other;
a first pair of guide channels defined by and extending through the first link;
a second pair of guide channels defined by and extending through the second link; and
a lining overlaying one or more of at least a portion of the joint structure, the first pair of guide channels, or the second pair of guide channels; and
a pair of actuation elements extending respectively through the first pair of guide channels, past the joint structure, and through the second pair of guide channels,
wherein the pair of actuation elements extend through the first pair of guide channels and the second pair of guide channels, and are configured to be placed in tension to thereby exert a force to control articulation of the first link and the second link relative to each other from a neutral, unarticulated state about the joint structure, and
wherein the lining provides an interior guide surface along at least part of a path of the pair of actuation elements extending through the first pair of guide channels, the second pair of guide channels, and past the joint structure.

2. The articulable mechanism of claim 1, wherein the lining comprises at least one insert fitted over at least the portion of the joint structure, the first pair of guide channels, or the second pair of guide channels.

3. The articulable mechanism of claim 1, wherein the lining overlays one or both of the first pair of guide channels or the second pair of guide channels, and at least the portion of the joint structure.

4. The articulable mechanism of claim 3, wherein the joint structure comprises a first joint feature fixed to the first link and a second joint feature fixed to the second link, and the lining overlays one or both of the first joint feature and the second joint feature.

5. The articulable mechanism of claim 4, wherein at least one of the first joint feature and the second joint feature comprises a hub structure with an outer surface along which the pair of actuation elements extend, and the lining overlays the hub structure.

6. The articulable mechanism of claim 1, wherein the lining is molded over at least the portion of the joint structure, the first pair of guide channels, or the second pair of guide channels.

7. The articulable mechanism of claim 1, wherein the lining is mechanically interlocked with at least the portion of the joint structure, the first pair of guide channels, or the second pair of guide channels.

8. The articulable mechanism of claim 1,
wherein the guide channels of the first pair of guide channels are on opposite sides of a first articulation axis of the first link about the joint structure, and
wherein the guide channels of the second pair of guide channels are on opposite sides of a second articulation axis of the second link about the joint structure.

9. The articulable mechanism of claim 1, wherein the lining is made of a different material than the joint structure, and the first and second links.

10. The articulable mechanism of claim 9, wherein the lining is made of a polymeric material.

11. The articulable mechanism of claim 1, wherein the joint structure comprises complementary rolling contact surfaces.

12. The articulable mechanism of claim 1, wherein the joint structure comprises complementary and intermeshing joint features.

13. The articulable mechanism of claim 1, wherein the joint structure is configured as a pinned joint.

14. A medical instrument comprising:
a shaft;
the articulable mechanism of claim 1 coupled to the shaft; and
an end effector coupled to the articulable mechanism.

15. A method of manufacturing an articulable mechanism, the method comprising:
coupling a first link of the articulable mechanism and a second link of the articulable mechanism to each other via a joint structure, the joint structure being configured to enable articulation of the first link and the second link relative to each other;
overlaying a lining over one or more of at least a portion of the joint structure, a first pair of guide channels defined by and extending through the first link, or a second pair of guide channels defined by and extending through the second link; and
extending a pair of actuation elements respectively through the first pair of guide channels, past the joint structure, and through the second pair of guide channels,
wherein the lining provides an interior guide surface along at least part of a path of the pair of actuation elements extending through the first pair of guide channels, the second pair of guide channels, and past the joint structure.

16. The method of claim 15, wherein overlaying the lining comprises fitting at least one insert over one or both of the first pair of guide channels or the second pair of guide channels, and over at least the portion of the joint structure.

17. The method of claim 15, wherein overlaying the lining comprises overlaying the lining over at least the portion of the joint structure.

18. The method of claim 17, wherein coupling the first link and the second link to each other via the joint structure comprises coupling a first joint feature fixed to the first link with a second joint feature fixed to the second link, and
wherein overlaying the lining over at least the portion of the joint structure comprises fitting at least one insert over one or both of the first joint feature and the second joint feature.

19. The method of claim 18, wherein at least one of the first joint feature and the second joint feature comprises a hub structure with an outer surface along which the pair of actuation elements extend, and
wherein overlaying the lining over one or both of the first joint feature and the second joint feature comprises overlaying the lining over the hub structure.

20. The method of claim 16, wherein overlaying the lining comprises:
molding a polymeric material over one or more of the at least a portion of the joint structure, the first pair of guide channels, or the second pair of guide channels, or
mechanically interlocking a polymeric part with one or more of the at least a portion of the joint structure, the first pair of guide channels, or the second pair of guide channels.
